(19) United States
(12) Patent Application Publication
(10) Pub. No.: US 2009/0105719 A1
(43) Pub. Date: Apr. 23, 2009

(54) PRECISION STENT POSITIONER

(75) Inventors: R. John D'A. Honey, Toronto (CA); Anthony D. Hammack, Bloomington, IN (US); Jimmy Lloyd Taylor, JR., Poland, IN (US)

Correspondence Address: BRINKS HOFER GILSON & LIONE/CHICAGO/COOK, PO BOX 10395, CHICAGO, IL, 60610, US

(73) Assignee: Vance Products Incorporated, d/b/a Cook Urological Incorporated, Spencer, IN (US)

(21) Appl. No.: 11/875,593

(22) Filed: Oct. 19, 2007

(51) Int. Cl. A61F 2/04

(52) U.S. Cl. 606/108; 623/23.7

(57) ABSTRACT

A positioner is provided that is able to precisely position a stent, such as a ureteral stent, by using an anatomical landmark, such as a ureteral orifice. The positioner is placed over a wire guide and advanced until the proximal portion of the stent abuts a stent-stop. The positioner and stent are together pushed until the positioner reaches the ureteral orifice. The stent can be deployed and the positioner can be removed leaving the stent correctly positioned within the kidney and bladder.

### FIELD OF THE INVENTION

The invention relates to medical devices, particularly those used in conjunction with positioning stents.

### BACKGROUND OF THE INVENTION

Indwelling ureteral stents have been widely used for years. Such stents are placed in the ureter, which is the duct between the kidney and the bladder, for the purpose of establishing and/or maintaining an open, patent flow of urine from the kidney to the bladder. Some reasons for placing a ureteral stent include extrinsic compression occlusions, ureteral injury due to trauma, and obstructive uropathy.

The typical ureteral stent can be composed of various radiopaque polymers, including polyethylene, silicone, polyurethane, and thermoplastic elastomer. These stents are retained in the ureter by a retentive anchoring means, such as a curve shape, pigtail, coil, J-shape, or hook configuration, at either end of the stent that engages the walls of the bladder and the kidney, respectively. The stent is resilient to allow it to be straightened for insertion into a body passageway and returned to its predetermined retentive anchoring shape when in situ.

Indwelling ureteral stents are positioned in the ureter by antegrade (percutaneous) placement, retrograde (cystoscopic) placement through the urethra, as well as by open ureterotomy or surgical placement in the ureter by direct manipulative control. Ureteral stent positioning has heretofore been accomplished by two basic methods.

In one method, a wire guide is introduced into the ureteral orifice in the bladder via a cystourethroscope under direct vision. The wire guide is advanced up the ureter until the advancing flexible tip of the guide is confirmed by X-ray or fluoroscopy to be in the renal pelvis of the kidney. A tubular stent with both ends open is fed onto the exposed external segment of the wire guide and advanced over the wire guide by hand until a short segment of the stent is visible outside the cystoscope. A pusher catheter (usually a length of tubing) is then fed onto the exposed external end of the wire guide and advanced over the wire guide by hand until it butts against the stent. With the wire guide held stationary, the positioner is advanced over the wire guide to push the tubular stent up the ureter to the renal pelvis. With the anatomically proximal end of the stent in the renal pelvis, the positioner is held stationary while the wire guide is gradually extracted from the stent and the positioner. It is desired that as the wire guide leaves the distal end of the tubular stent, the retentive hook or curve of the distal end of the stent is formed to retain the stent in the pelvis of the kidney, and as the wire guide is withdrawn past the proximal, or intravesicle, end of the stent, the retentive hook or curve of the proximal end is formed so that the stent end is retained within the bladder. However, often times the stent is placed too far into the kidney or not far enough due to physician inexperience, anatomical challenges, the inability to properly visualize the stent's progression through the bodily passage, etc. Improper placement leads to poor stent drainage and often the need to reposition the stent subjecting the patient to the possibility of further bodily injury and infection from multiple invasive procedures. Accordingly, proper stent placement is difficult to achieve.

In another method of ureteral stent placement, a ureteral stent having one end closed is backloaded onto a wire guide. In this “push-up” method, the tip of the wire guide contacts the closed end of the ureteral stent, which is then introduced into the ureteral orifice in the bladder via a cystourethroscope under direct vision. The stent is advanced up the ureter under fluoroscopic control until the tip of the stent lies within the renal pelvis. A positioner catheter or length of tubing is fed onto the external end of the wire guide and advanced over the wire guide by hand until it butts against the open, distal end of the stent. The positioner is held steady while the wire guide is removed in a fashion similar to that described above. Like the previous method described, this method, too, suffers from the same drawbacks and often results in a poorly positioned stent.

What is needed is a device for insuring the proper placement of a stent that overcomes the limitations known in the art.

### BRIEF SUMMARY OF THE INVENTION

A medical device is provided that includes an elongated tubular body having a proximal portion, a distal portion, and a lumen extending therethrough, a taper configured near the distal portion of the elongated tubular body, wherein an outer diameter of the taper is larger than an anatomical landmark, and a stent-stop configured near the distal portion of the elongated tubular body, wherein the stent-stop is configured to receive a proximal portion of a stent.

In addition, an insertion device is provided that includes a bit having a proximal and distal portion, a hand collet, wherein the hand collet is adapted to receive the proximal portion of the bit, and further wherein the distal portion of the bit is adapted to receive a sent-stop.

Furthermore, a method for deploying a stent that includes inserting a wire guide to the location of a stent deployment, placing a stent, having a first anchor at the proximal portion and a second anchor at the distal portion, onto the wire guide, providing a positioner having a stent-stop and a taper, wherein the taper has a diameter larger than an anatomical landmark, abutting the proximal portion of the stent to the stent-stop, pushing the positioner until the taper reaches the anatomical landmark, withdrawing the wire guide to deploy the second anchor, and withdrawing the wire guide and positioner to deploy the first anchor.

### BRIEF DESCRIPTION OF THE SEVERAL VIEWS OF THE DRAWINGS

The embodiments will be further described in connection with the attached drawing figures. Throughout the specification, like reference numerals and letters refer to like elements. It is intended that the drawings included as a part of this specification be illustrative of the embodiments and should in no way be considered as a limitation on the scope of the invention.

FIG. 1 is an exemplary anatomical view of a human bladder and kidney;

FIG. 2 is a perspective view of a first embodiment of a positioner;

FIG. 3 is a cross sectional view of a first embodiment of a positioner;

FIG. 4 is a perspective view of an embodiment of a retention disk;

FIG. 5 is a top view of an embodiment of a retention disk;

FIG. 6 is a perspective view of an embodiment of a retention disk insertion device;

FIG. 7 is a cross sectional view of a first embodiment of a positioner with a wire guide and stent partially disposed therein;

FIG. 8 is a first embodiment of a positioner depicting a use of the device;

FIG. 9 is a first embodiment of a positioner depicting a use of the device;

FIG. 10 is a perspective view of a second embodiment of a positioner;

FIG. 11 is a cross sectional view of a retention cap;

FIG. 12 is a cross sectional view of a second embodiment of a positioner;

FIG. 13 is a cross sectional view of a second embodiment of a positioner with a wire guide and stent partially disposed therein;

FIG. 14 is another embodiment of a positioner with a wire guide and stent partially disposed therein;

FIG. 15 is cross-sectional view of an embodiment of a positioner with a retention cap disposed thereon; and

FIG. 16 is a flow chart depicting a method of using the device.

### DETAILED DESCRIPTION OF PRESENTLY PREFERRED EMBODIMENTS

The embodiments provide an apparatus that is able to properly position a stent within a kidney and a bladder. However, it is contemplated that which is disclosed herein can be used to place stents in other parts of the body, including but not limited to, the urethra, the vascular system, and the pancreatic-biliary system. Moreover, that which is disclosed herein in not limited to use in human beings.

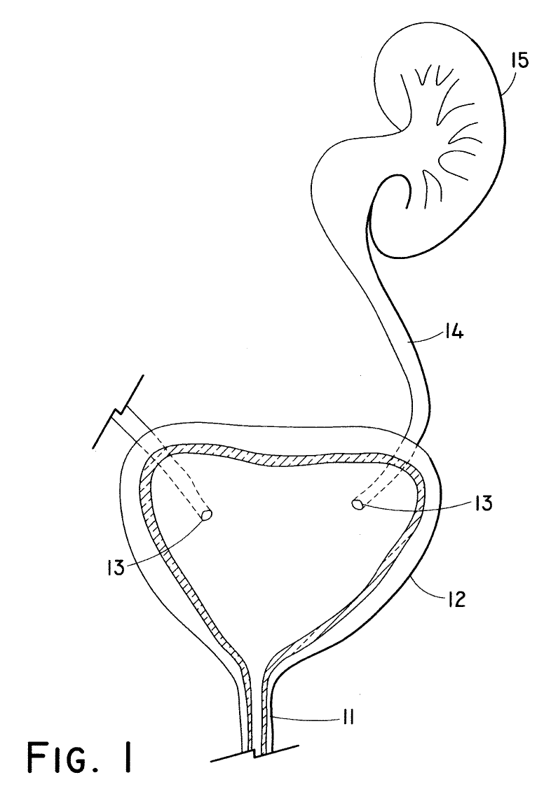

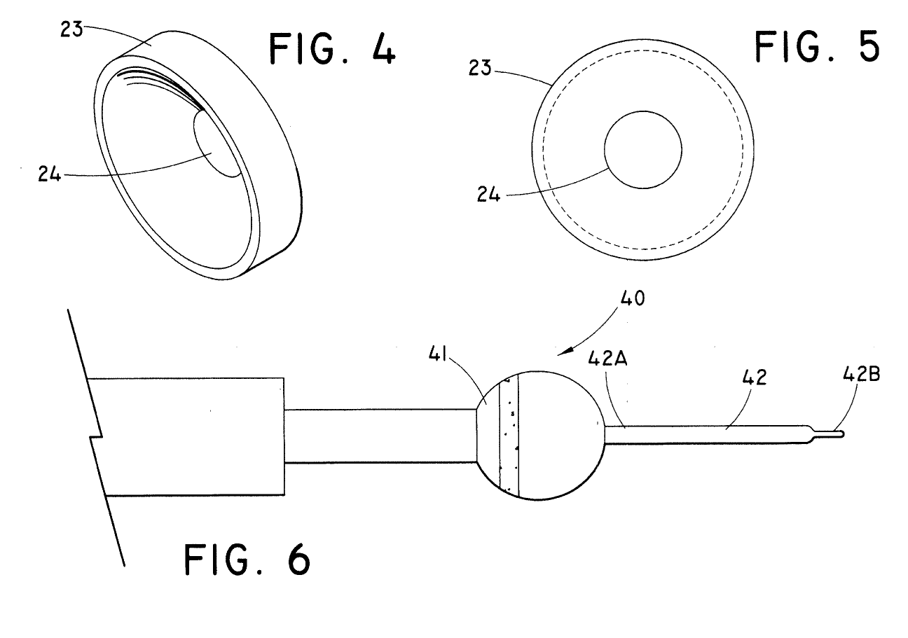

A more detailed description of the embodiments will now be given with reference to FIGS. 1-16. The present invention is not limited to those embodiments illustrated; it specifically contemplates other embodiments not illustrated but intended to be included in the claims.

As depicted in FIG. 1, the average human has urethra 11 that is attached to bladder 12. The average human adult urethra is about 9 to 10 mm wide. Bladder 12 connects to the ureter 14 at the ureteral orifice 13. Ureter 14 is connected to kidney 15. The average human adult ureteral orifice is about 2 mm wide (6 Fr.) to 3 mm wide (9 Fr.), and the average human child ureteral orifice is about 1.5 mm (4.5 Fr.) to 2.0 mm (6 Fr.).

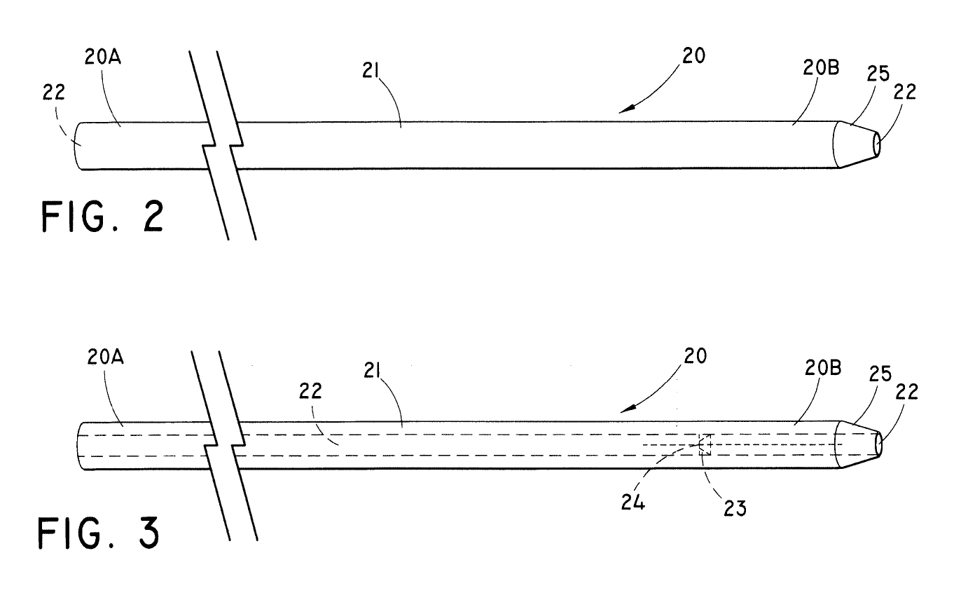

FIGS. 2 and 3 depict an exemplary embodiment of a positioner 20 having proximal portion 20A, distal portion 20B, and lumen 22 extending throughout elongated tubular body 21. Positioner 20 is used to position a stent within a kidney and bladder by using an anatomical landmark such as a ureteral orifice rather that requiring the medical professional to position the stent by feel and visualization means alone. Thus, by using an anatomical landmark, a medical professional is better apt to position a ureteral stent in what would otherwise be a very difficult procedure. In short, a wire guide is fed up to kidney 15, a ureteral stent is loaded onto the wire guide, the proximal portion of the ureteral stent is positioned so as to abut the positioner, the positioner is pushed until it reaches ureteral orifice 13, the wire guide is removed from the distal portion of the stent leaving it within kidney 15; finally, the wire guide and positioner are removed leaving the proximal portion of the stent dwelling within bladder 12.

Positioner 20 has taper 25 located at the distal portion 20B of elongated tubular body 21 and is a blunt tip formed using a heated glass mold, heated metal alloy mold, or by other methods known in the art, such as buffing, grinding, or using a heat shrinkable tubing as a means to form a taper. Taper 25 is larger than ureteral orifice 13 but small enough to fit through urethra 11. Taper 25 is approximately 3-5 mm long, although other sizes are contemplated depending upon the needs of the patient. Thus, taper 25 can be sized to fit an average patient or any particular patient. Ureteral orifice is able to stretch; thus, it is desired that no more than a minimal portion of taper 25 enter ureteral orifice, because otherwise, the anatomical landmark used to position stent 26 could inadvertently be passed.

Elongated tubular body 21 is an 18 Fr. polyurethane tube that is approximately 30-40 cm long, although other sizes are contemplated depending upon the needs of the patient. In addition, elongated tubular body 21 can be made from other materials, including but not limited to polytetrafluoroethelyne (PTFE), common medical polymers such as polyethylene, polypropylene, silicone, stainless steel, tungsten, Titanium, PEEK, brass, aluminum, nylons, vinyls (such as PVC), and other medically acceptable metal materials. Moreover, elongated tubular body 21 may be covered with a coating to ease friction; such coatings include but are not limited to a hydrophilic coating, poly vinyl alcohol, poly vinyl pyrrolidone, hydrophobic coating (such as parylene), anti-microbial or antiseptic coating, Teflon, and other medically acceptable coatings.

Additionally, elongated tubular body 21 may be coated with a bioactive agent. As used herein, “bioactive agent” refers to any substance that can be used for therapeutic, prophylactic, or diagnostic purposes. A therapeutic purpose refers to the treatment of an on-going disease or disorder—the goal being to cure it or at least ameliorate its symptoms. A prophylactic purpose refers to the administration of a bioactive agent before any disease or disorder has manifested itself or to administration after the disease or disorder has been subjected to therapeutic treatment to prevent recurrence of the disease or disorder or of symptoms of the disease or disorder. Elongated tubular body 21 may be coated with, formed with, or impregnated with a fluoropolymer or other protective, lubricious coating and/or a bioactive agent selected to mitigate or eliminate encrustation with long-term implantation of medical devices. Heparin or other drug-containing coatings may be applied to elongated tubular body 21 by any suitable means, including spraying, dipping, solvent casting, and the like. Fluoropolymers, such as PTFE, help to enable the bonding of certain drugs, such as heparin, to the surface of elongated tubular body 21. Other drugs useful for mitigating or preventing encrustation include heparin, covalent heparin, dexamethazone, dexamethasone sodium phosphate, dexamethasone acetate, and other dexamethasone derivatives, triclosan, silver nitrate, ofloxacin, ciproflaxin, phosphorylcholine, and triemethoprim. In addition, one or more bioactive agents may be placed on the surface of, or contained within, elongated tubular body 21 in order to assist in patient care and comfort. For instance, an antimicrobial drug, such as a combination of rifampin and minocycline, may help to reduce inflammation and microbial activity in the vicinity of the stent. Antimicrobial coatings applied to elongated tubular body 21 may include the following drugs or their salts or derivatives: rifampin, minocycline, a mixture of rifampin and minocycline, a non-steroidal anti-inflammatory agent, a penicillin, a cephalosporin, a carbepenem, a beta-lactam, an antibiotic, an aminoglycoside, a macrolide, a lincosamide, a glycopeptide, a tetracyline, a chloramphenicol, a quinolone, a fucidin, a sulfonamide, a trimethoprim, a rifamycin, an oxaline, a streptogramin, a lipopeptide, a ketolide, a polyene, an azole, an echinocandin, alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, rifampycin, silver, benzyl peroxide, alcohols, and carboxylic acids and salts, and silver sulfadiazine. Also useful as antimicrobials are anthracyclines, such as doxorubicin or mitoxantrone, fluoropyrimidines such as 5-fluoroacil, and also podophylotoxins, such as etoposide. The salts and the derivatives of all of these are meant to be included as examples of antimicrobial drugs. Analgesics, such as aspirin or other non-steroidal anti-inflammatory drugs, may also be applied to elongated tubular body 21 to reduce pain and swelling upon implantation. These drugs or their salts or derivatives may include aspirin and non-steroidal anti-inflammatory drugs, including naproxen, choline, diflunisal, salsalate, fenoprofen, flurbiprofen, ketoprofen, ibuprofen, oxaprozin, diclofenac, indomethacin, sulindac, acetoaminophen, tolmetin, meloxicam, piroxicam, meclofenamate, mefanimic acid, nabumetone, etodelac, keterolac, celecoxib, valdecoxib, and rofecoxib, mixtures thereof, and derivatives thereof. Other analgesics or anesthetics that may be coated onto the surface of elongated tubular body 21 include opioids, synthetic drugs with narcotic properties, and local anesthetics to include at least paracetamol, bupivacaine, ropivacaine, lidocaine, and novacaine, alfentanil, buprenorphine, carfentanil, codeine, codeinone, dextropropoxyphene, dihydrocodeine, endorphin, fentanyl, hydrocodone, hydromorphone, methadone, morphine, morphinone, oxycodone, oxymorphone, pethidine, remifantanil, sulfentanil, thebaine, and tramadol, mixtures thereof, and derivatives thereof. Any of these bioactive agent coatings can be applied in a time-release manner should there be a need for positioner to dwell within the patient for an extended period of time. Other bioactives include but are not limited to those discussed in U.S. patent application Ser. No. 10/410,587, filed Apr. 8, 2003 and incorporated herein by reference in its entirety.

It is desired, although not required, that positioner 20 be long enough to reach ureteral orifice 13. Elongated tubular body 21 has lumen 22 extending throughout that has an inner diameter of approximately 0.115-0.119 inches, although other sizes are contemplated depending upon the needs of the patient. Disposed within elongated tubular body 21 is retention disk 23.

Turning to FIGS. 4, 5, and 7, retention disk 23 is a machined insert that serves to locate wire guide 27 in lumen 22 and also provides a means for advancing stent 26 along wire guide 27 by providing a stent-stop for proximal portion 26A of stent 26. Retention disk 23 can be made from any material that is hard enough to withstand the force of stent 26 pushing back on it, including but not limited to nylon, polytetrafluoroethelyne (PTFE), common medical polymers, polyurethanes, stainless steel, tungsten, Titanium, PEEK, brass, aluminum, and other medically acceptable metal materials. In addition, it is preferable, although not required that the material be radiopaque. The insert may be produced using injection molding for polymeric materials. Polymeric materials can be produced using traditional metal working tools such as a lathe or mill. Metallic parts could also be metal injection molded. Retention disk 23 has an outer diameter of about 0.120-0.124 inches and can be inserted into elongated tubular body 21 using insertion tool 40 depicted in FIG. 6.

As depicted in FIG. 6, proximal portion 42A of insertion tool 40 comprises a hand collet 41 that is used to hold bit 42. Retention disk 23 slides onto distal portion 42B of bit 42 and is pushed into elongated tubular body 21. Insertion tool 40 is then removed. Retention disk 23 is held in place by an interference fit with the inner surface of elongated tubular body 21 and/or a medical-grade glue. The length of bit 42 determines the depth of placement of retention disk 23. Here, retention disk 23 is placed at a depth of about 2 cm; however, the depth may vary depending upon the length of the stent's anchoring means. It is desired that the stent-stop be placed at a distance from the distal-most end of the positioner such that the placement distance is equal to the length of the proximal portion of the stent desired to remain with bladder 12. For example, if it is preferred that about 2 cm of the proximal portion of the stent remain within the bladder, then the stent-stop should be placed back about 2 cm from the distal-most end of the positioner, and the taper should be placed 2 cm distally from the stent-stop. Therefore, once the taper reaches the ureteral orifice, about 2 cm of stent will remain within bladder 12.

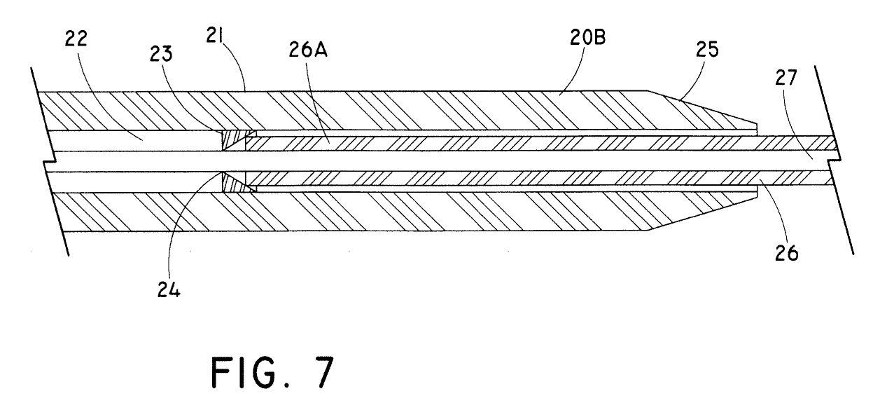

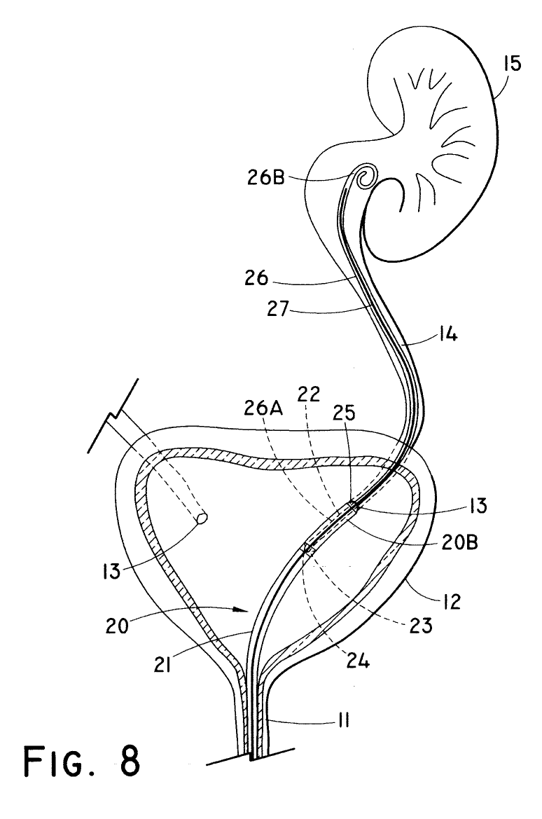

Turning to FIGS. 7 and 8, wire guide 27 is placed into kidney 15. Proximal portion 26A of stent 26, having an anchoring means, is loaded onto wire guide 27. Positioner 20 is placed over wire guide 27 such that wire guide 27 travels through lumen 24 of retention disk 23 and proximal portion 26A of stent 26 abuts retention disk 23. Lumen 24 of retention disk has a diameter of about 0.50 inches; however, other sizes can be used depending upon the needs of the patient and the diameter of the wire guide to be inserted therethrough. By pushing positioner 20, stent 26 is pushed up through urethra 11 and bladder 12. Positioner 20 is pushed until taper 25 of positioner 20 abuts ureteral orifice 13. Because taper 25 is larger than ureteral orifice 13, positioner 20 stops, indicating that stent 26 is in proper position for deployment.

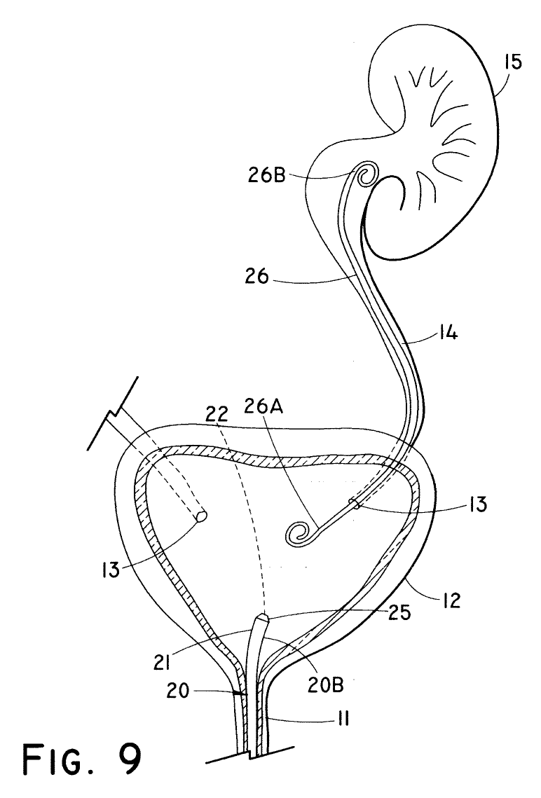

As shown in FIG. 8, stent 26 is deployed by withdrawing wire guide 27 from distal portion 26B of stent 26 causing distal portion 26B of stent 26 to form the anchoring means. Wire guide 27 and positioner 20 are withdrawn causing proximal portion 26A of stent 26 to form into the anchoring means as depicted in FIG. 9.

Furthermore, positioner 20 may further include any number of markers (not shown) that are visible under fluoroscopy means, X-Ray means, ultrasonic means, or other means known in the art, to aid in the placement of the stent. Markers can be made from Platinum-Iridium alloy or any other radiopaque material, such as gold or tungsten, or echogenic material. An echogenic material includes surface irregularities that reflect ultrasonic waves and thus, allow the material to be seen with ultrasonic imaging devices. Echogenic techniques are described in U.S. Pat. No. 5,081,997 and U.S. Pat. No. 5,289,831, assigned to the assignee of the present invention, and they are hereby incorporated by reference in their entirety.

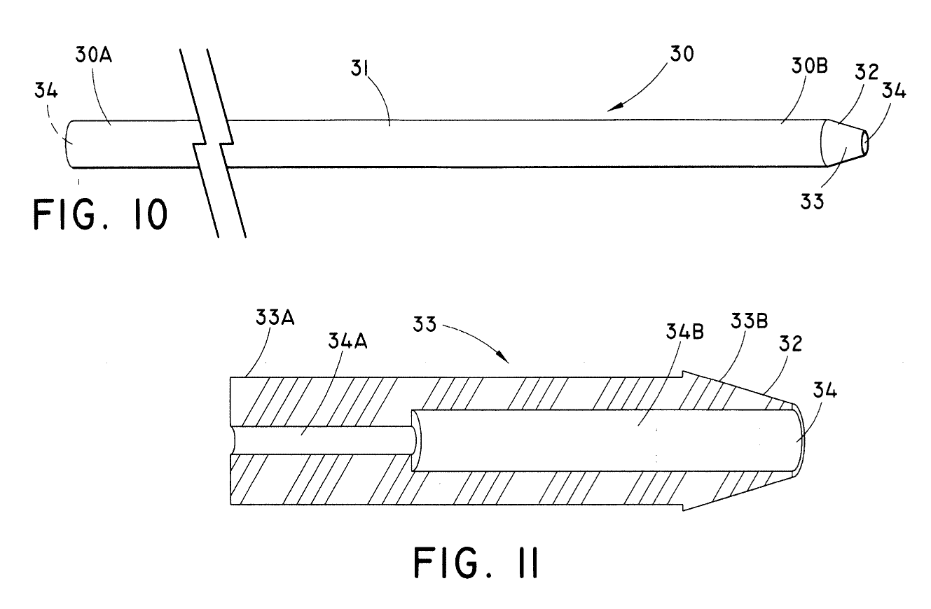

FIG. 10 depicts another embodiment of a positioner 30. Positioner 30 includes an elongated tubular body 31, a proximal portion 30A, and a distal portion 30B. Positioner 30 is like positioner 20, but instead of having a retention disk and taper formed from elongated tubular body, positioner 30 includes retention cap 33 as depicted in FIG. 11. Retention cap 33 serves as both the stop for proximal portion 26A of stent 26 as well as the tapered device tip. Retention cap 33 can be made from a soft polymeric material including, but not limited to common medical polymers, such as nylon, polyethylene, polypropylene, polyurethanes, vinyl, silicone, as well as metals and other medically accepted materials. Positioner 30 may also include any number of markers, as described above.

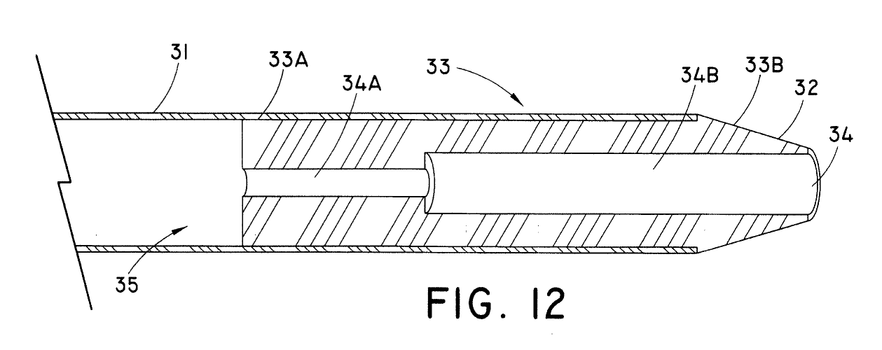

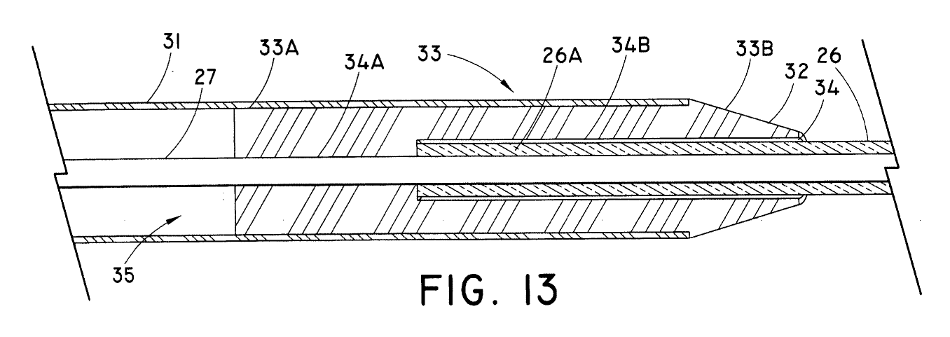

As depicted in FIG. 12, retention cap 33 is pushed into lumen 35 of elongated tubular body 31 and held in place by an interference fit with the inner surface of elongated tubular body 31 and/or a medical-grade glue. Retention tip 33 can be injection molded, insert molded, or molded via other methods known in the art. Retention cap 33 is about 2.2896 inches long, and the length of the tapered portion is about 0.394 inches. Retention cap 33 has lumen 34 extending throughout; however, the diameter of lumen 34 changes so as to provide a stent-stop for proximal portion 26A of stent 26. For example, the diameter of distal portion of lumen 34B is about 0.084 inches, whereas the diameter of proximal portion of lumen is about 0.0420 inches.

As depicted in FIG. 13, stent 26 is placed over wire guide 27. Wire guide 27 and proximal portion 26A of stent 26 are placed into positioner 30 such that proximal portion 26A of stent 26 abuts against smaller lumen 34A of retention cap 34. Thus, proximal portion 26A of stent 26 resides disposed within distal portion 34B of retention cap lumen 34. Positioner 30, along with stent 26, are pushed up through urethra 11 and bladder 12 until taper 32 of positioner 30 abuts ureteral orifice 13. Stent 26 is deployed as previously described.

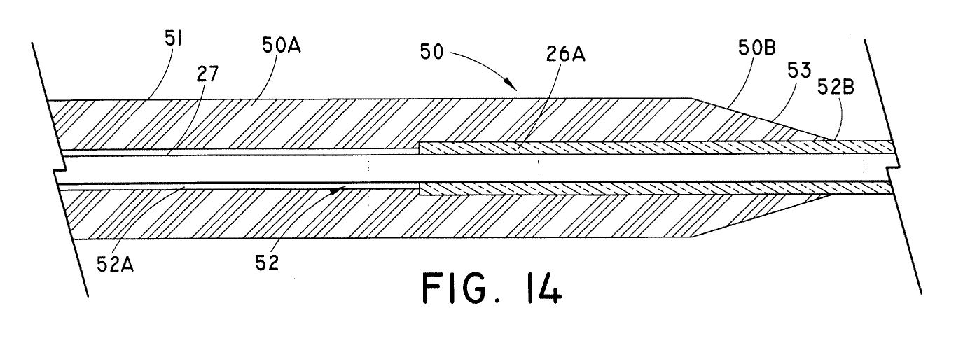

FIG. 14 provides another embodiment of a positioner 50 that includes proximal portion 50A, distal portion 50B, and lumen 52 extending throughout elongated tubular body 51. Positioner 50 is like positioner 20, however, instead of using a retention disk to form a stop for proximal portion 26A of stent 26, elongated tubular body 21 is manufactured with lumen 52 having two different diameters 52A, 52B. Elongated tubular body 51 could be manufactured by methods including but not limited to, using a heated glass mold, insert molding, injection molding, butt welding of pre-formed tubing, as well as by other methods known in the art. Larger lumen 52B has a diameter larger than the outer diameter of stent 26. Smaller lumen 52A is sized such that it is too small for stent 26 to fit therethrough, but it is still large enough for wire guide to fit through. Accordingly, the point where larger lumen 52B and smaller lumen 52A meet provides a stent-stop. Positioner 50 also includes taper 53 that has an outer diameter that is larger than ureteral orifice 13 but smaller than urethra 11. Positioner 50 may also includes any number of markers, as described above.

To use positioner 50, stent 26 is placed over wire guide 27. Wire guide 27 and proximal portion 26A of stent 26 are placed into positioner 50 such that proximal portion 26A of stent 26 abuts against smaller lumen 52A of elongated tubular body 51. Positioner 50, along with stent 26, are pushed up through urethra and bladder 12 until taper 53 of positioner 50 abuts ureteral orifice 13. Stent 26 is then deployed as previously described.

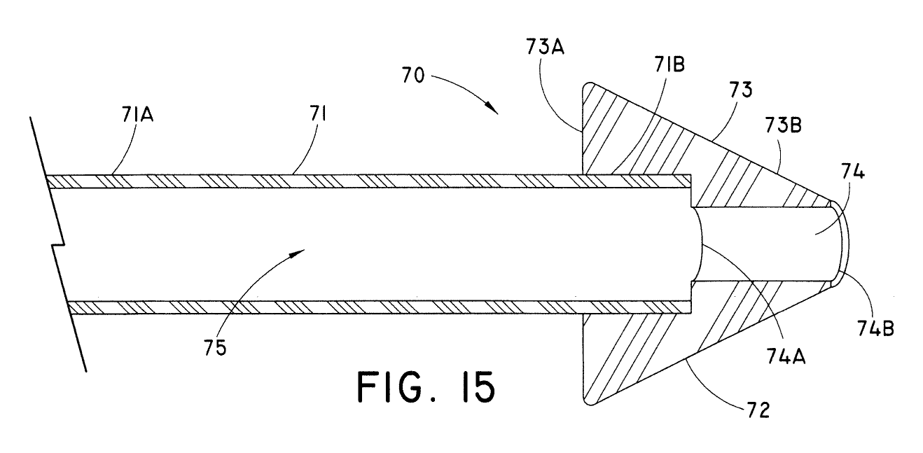

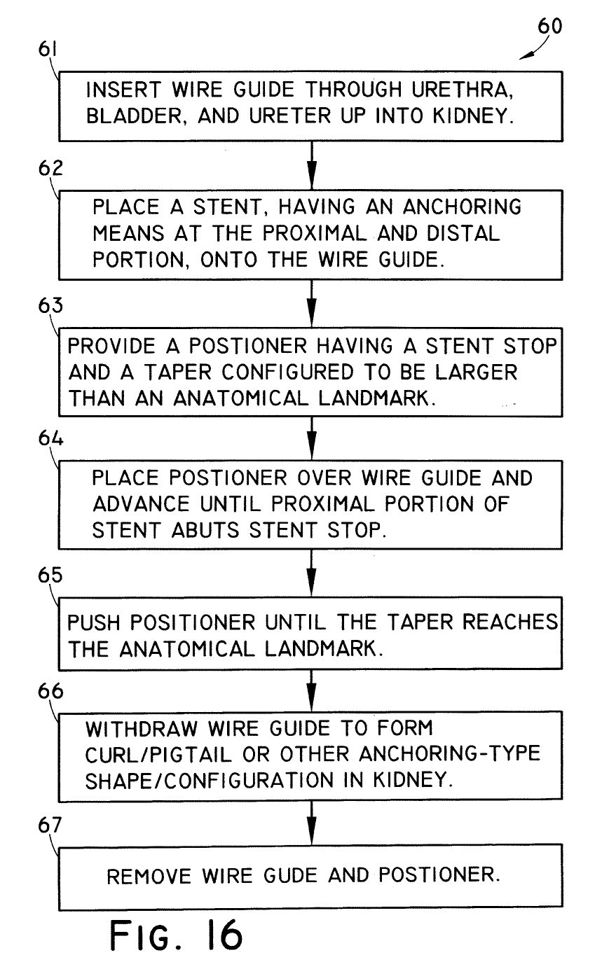

FIG. 15 is an alternate embodiment of FIG. 12, wherein retention cap 73 is disposed upon elongated tubular body 71. Positioner 70 includes elongated tubular body 71 that has lumen 75 extending throughout. Attached to distal portion 71B of elongated tubular body 71 is retention cap 73. Retention cap 73 is like that depicted in FIG. 12, however, retention cap 73 fits over elongated tubular body 71 rather than in it. Retention cap has taper 72, like the other embodiments, and lumen 74 extending throughout with two different lumenal diameters—wider portion 74B that changes to narrower portion 74A that provides a stent-stop. Thus, a wire guide (not shown) is able to extend through retention cap 73 and through proximal portion 71A of elongated tubular body 71. In addition, a proximal portion of a stent (not shown) is able to be inserted into distal portion 73B of retention cap 73 until it abuts narrow lumen 74A at proximal portion 73A of retention cap 73. Positioner 71 is able to be pushed until taper 72 reaches the ureteral orifice (or other anatomical landmark). The stent can be deployed as previously described.

FIG. 16 depicts a method of deploying a stent using a positioner 60. A wire guide is inserted through the urethra, bladder, and ureter up into kidney 61. A stent, having an anchoring means at the proximal and distal portions, is placed onto the wire guide 62. A positioner is provided having a stent-stop and a taper configured to be larger than an anatomical landmark 63. The positioner is placed over the wire guide and advanced until the proximal portion of the stent abuts the stent-stop 64. The positioner is pushed until the taper of the positioner reaches an anatomical landmark, such as a ureteral orifice 65. The wire guide is withdrawn, and the distal anchoring means forms within the kidney 66. The wire guide and positioner are removed, leaving the proximal anchoring means to form within the bladder 67.

As is evident, the embodiments provide a very effective solution for positioning a stent. The foregoing description and drawings are provided for illustrative purposes only and are not intended to limit the scope of the invention described herein or with regard to the details of its construction and manner of operation. In addition, the dimensions and sizes described herein are not intended to be limiting as they can be altered to fit the needs of the patient or medical professional. Moreover, the positioner is not limited for use with a ureteral stent or the use of the ureteral orifice as an anatomical landmark. It will be evident to one skilled in the art that modifications and variations may be made without departing from the spirit and scope of the invention. Changes in form and in the proportion of parts, as well as the substitution of equivalents, are contemplated as circumstances may suggest and render expedience; although specific terms have been employed, they are intended in a generic and descriptive sense only and not for the purpose of limiting the scope of the invention set forth in the following claims.

What is claimed is:

1. A medical device comprising:
an elongated tubular body having a proximal portion, a distal portion, and a lumen extending therethrough;
a taper configured near the distal portion of the elongated tubular body, wherein an outer diameter of the taper is larger than an anatomical landmark; and
a stent-stop configured near the distal portion of the elongated tubular body, wherein the stent-stop is configured to receive a proximal portion of a stent.

2. The device of claim 1, wherein the stent-stop is selected from the group consisting of a retention disk, a retention cap, and a change in lumenal diameter.

3. The device of claim 1, wherein the lumen has a distal portion and a proximal portion;
wherein the proximal portion of the lumen is adapted to receive a wire guide; and
wherein the distal portion of the lumen is adapted to receive a wire guide and the proximal portion of the stent.

4. The device of claim 1, wherein the anatomical landmark is a ureteral orifice.

5. The device of claim 1, wherein the taper is configured from the elongated tubular body.

6. The device of claim 1, wherein the elongated tubular body further comprises a coating.

7. The device of claim 1, wherein the elongated tubular body is configured to fit through a urethra.

8. The device of claim 1, wherein the stent is a ureteral stent.

9. The device of claim 1, wherein the stent-stop is configured in a direction proximally from the taper.

10. The device of claim 1, further comprising at least one marker in communication with at least one of the elongated tubular body, the stent-stop, and the taper.

11. The device of claim 1, wherein the stent-stop is a retention cap and further wherein the taper is integrated into the retention cap.

12. The device of claim 1, wherein the stent-stop is a retention disk and further wherein a distal portion of the retention disk is adapted to receive the proximal portion of the stent.

13. An insertion device comprising:
a bit having a proximal and distal portion;
a hand collet, wherein the hand collet is adapted to receive the proximal portion of the bit, and further wherein the distal portion of the bit is adapted to receive a sent-stop.

14. The device of claim 13, wherein the bit is configured for insertion into an elongated tubular body.

15. A method for deploying a stent comprising;
inserting a wire guide to the location of a stent deployment;
placing a stent, having a first anchor at the proximal portion and a second anchor at the distal portion, onto the wire guide;
providing a positioner having a stent-stop and a taper, wherein the taper has a diameter larger than an anatomical landmark;
abutting the proximal portion of the stent to the stent-stop;
pushing the positioner until the taper reaches the anatomical landmark;
withdrawing the wire guide to deploy the second anchor; and
withdrawing the wire guide and positioner to deploy the first anchor.

16. The method of claim 15, wherein the stent is a ureteral stent.

17. The method of claim 15, wherein the anatomical landmark is a ureteral orifice.

18. The method of claim 15, wherein the location for stent deployment is a ureter.

19. The method of claim 15, wherein the stent-stop is selected from the group consisting of a retention disk, a retention cap, and a change in lumenal diameter.

20. The method of claim 15, wherein at least one of the first and second anchor is a curl or pigtail.
